(19) United States
(12) Patent Application Publication
(10) Pub. No.: US 2023/0115099 A1
(43) Pub. Date: Apr. 13, 2023

(54) COMBINATION OF CURCUMINOIDS WITH KRILL OIL FOR IMPROVED DELIVERY AND EFFICACY

(71) Applicant: First Endurance, LLC, Sandy, UT (US)

(72) Inventor: Luke Robert Bucci, Reno, NV (US)

(21) Appl. No.: 17/881,145

(22) Filed: Aug. 4, 2022

Related U.S. Application Data
(60) Provisional application No. 63/229,703, filed on Aug. 5, 2021.

(51) Int. Cl. A23L 33/115; A23L 33/105

(52) U.S. Cl. CPC A23L 33/115; A23L 33/105

(57) ABSTRACT

Supplements including krill oil, including significant fractions of phosopholipids (e.g., phosphatidylcholine (“PC”)), as well as triglycerides, in combination with curcuminoids. The particular formulation of PC and curcuminoids as packaged herein, in a rigid LiquidCap form, rather than a flexible softgel capsule, so as to purposefully exclude glycerin, results in the formation of a PC:curcuminoid complex, which provides for easier manufacturing, less incidence of capsule leakage, and perhaps most importantly, better bioefficacy. The supplements can be used to reduce joint and other inflammation, improve brain health and brain function, reduce risk of heart disease, treat delayed onset muscle soreness from exercise, exercise-induced muscle damage from over-exercise, muscular pain after intense exercise, musculotendinous pain after strenuous exercise (endurance and/or resistance exercise), joint pain after intense strenuous exercises, and/or to provide improvements in mood, cognition, cardiovascular functions (improved circulation, efficient cardiac performance/rhythm and other benefits of omega-3s and curcuminoids.

### CROSS-REFERENCE TO RELATED APPLICATIONS

This application claims priority to and the benefit of U.S. Provisional Patent Application No. 63/229,703, filed on Aug. 5, 2021, which is hereby incorporated by reference in its entirety.

### BACKGROUND OF THE INVENTION

### 1. The Field of the Invention

The present invention relates to nutraceutical formulations, particularly to formulations providing both krill oil and curcuminoids.

### 2. Description of Related Art

Krill oil, which is a source of docosahexaenoic acid (DHA) and eicosapentaenoic acid (EPA), can be derived or otherwise obtained from Antarctic or other species of krill. Krill oil is rich in omega-3 fatty acids, including DHA and EPA, and has been used as a dietary supplement, and may be associated with numerous health benefits, including, but not limited to decreased swelling or inflammation, lower cholesterol, reduced risk of formation of undesirable blood clots, reduced risk of heart disease, improved brain function, and improved eye health.

While omega-3 fatty acids are available from oils derived or obtained from various species of fish (e.g., available in the market as fish oil), some studies indicate that the fatty acids available in krill oil may be more readily available to the body, as compared to other fish oil supplements. Such may be due in part to fatty acids in fish oil being typically in the form of triglycerides, while in krill oil, a significant fraction of such omega-3 fatty acids are present as phospholipids.

In addition to krill oil, curcumin is another supplement touted as providing various health benefits. For example, some research seems to indicate that curcumin may be beneficial in the treatment of age related neurodegenerative diseases, such as Alzheimer's disease (e.g., see Maiti et al., Use of Curcumin, a Natural Polyphenol for Targeting Molecular Pathways in Treating Age-Related Neurodegenerative Diseases, Int J. Mol Sci. 2018 19(6):1637, herein incorporated by reference in its entirety), cancer prevention, and other benefits, although significant problems exist with absorption, uptake and bioavailability of curcuminoids. In particular, curcuminoids are difficult to absorb and exhibit low bioavailability due to the inherent instability of curcuminoids, and poor solubility of such compounds in typical body fluids, so that curcuminoids are not efficiently absorbed through the gastrointestinal tract, but often tend to degrade and be metabolized (e.g., in the liver or elsewhere) to forms exhibiting low bioavailability, eventually being excreted in the urine and/or feces, without providing the promised benefits.

While krill oil supplements, and even supplements including both krill oil and curcumin together are available within the art, difficulties remain with absorption, uptake, and bioavailability, particularly with respect to the curcuminoid portion of such formulations. In addition, existing blends including both krill oil and curcumin (e.g., such as available from Swanson, packaged in a softgel capsule) exhibit challenges associated with manufacture and capsule packaging (e.g., capsule leakage) in addition to the generally low absorption, uptake, and bioavailability of the supplement components (particularly the curcumin portion of the supplement). While significant research has been devoted to mechanisms for improving absorption, uptake and bioavailability, such problems remain largely unaddressed. The present invention is directed to improvements that address such issues.

### BRIEF SUMMARY OF THE INVENTION

The present invention is directed to supplements that include both krill oil and curcuminoids in an oily liquid phospholipid/triglyceride matrix, substantially void of free glycerin or similar polyol plasticizers (e.g., commonly used in softgel capsule packing to aid in sealing of the softgel, and keep the softgel capsule from cracking). A significant fraction of the omega-3 fatty acids in the krill oil are advantageously present in the form of phospholipids (e.g., particularly phosphatidylcholine, with carbon chain lengths of greater than 18 carbon atoms, such as 20 or 22 carbons) rather than as just triglycerides, which is the case for other conventional fish oil. Preferably, the omega-3 fatty acids of krill oil phospholipids are on the sn-2 position (middle carbon atom) of the glycerol backbone of each phospholipid molecule. Krill oil phospholipids also contain saturated fatty acids, usually palmitic acid, on the sn-1 position (one of the end carbons of the glycerol backbone), with the phosphate group on the other end carbon (sn-3).

In the absence of free glycerin or similar plasticizers necessarily present in softgels, the curcuminoid is advantageously able to form a weak, non-covalently bonded complex (e.g., held together by Van der Waals forces and hydrogen bonding) between the phosphatidylcholine (“PC”) and curcuminoid. Such complexes are only believed to be capable of formation due to the absence of free glycerin and similar polyol plasticizers, which are of necessity present in softgel capsules. The Swanson supplement and other similarly described supplements packaged in a softgel capsule are not void of glycerin, and are thus not capable of forming the desired complex. For example, existing softgel capsules are formed from gelatin (e.g., kosher gelatin), glycerin (e.g., Non-GMO vegetable glycerin), and water, providing a flexible, soft capsule. While perhaps convenient for the consumer to take orally, such capsules contribute to the low bioavailability, absorption, and uptake of curcuminoid supplements, and such existing softgel capsules exhibit problems with leakage.

Rather than employing such softgel packaging, the present invention employs a rigid liquid capsule packaging, that does not require the inclusion of any glycerin or similar plasticizer that would interfere with the desired formation of the PC-curcuminoid complex. For example, one embodiment of the present invention is directed to a supplement including krill oil including phosphatidylcholine, and one or more curcuminoids, in a triglyceride matrix. The krill oil phosphatidylcholine and one or more curcuminoids are present as weakly non-covalently bonded complexes, held together with Van der Waals forces between the fatty acids of the krill oil phospholipids and the hydrophobic, alkenyl regions of curcumin and/or hydrogen bonding between the phospholipid phosphate group and curcumin hydroxyl groups, wherein the supplement is packaged in a capsule that is not a softgel capsule, the capsule being void of glycerin and other polyol plasticizers.

Another embodiment is directed to methods of treatment, using such a supplement, to treat inflammation (e.g., inflammation of joints or other tissues), by orally administering such a supplement to the person being treated. Such supplements may also be used to treat delayed onset muscle soreness from exercise, exercise-induced muscle damage from over-exercise, muscular pain after intense exercise, musculotendinous pain after strenuous exercise (endurance and/or resistance exercise), and/or joint pain after intense strenuous exercises. In addition to such various treatments, the present supplements may be useful for improvements in mood, cognition, cardiovascular functions (improved circulation, efficient cardiac performance/rhythm and other benefits of omega-3s and curcuminoids.

In an embodiment, the phosphatidylcholine-curcuminoid complex that forms includes a structure where the relatively rigid curcuminoid molecule is entrapped within a long-chain omega-3 phosphatidylcholine phospholipid, shielding the curcuminoid from adverse interactions with hydrophilic, aqueous and/or metabolic environments. Rather than being absorbed into the portal blood vessels for transport and metabolism by the liver, and subsequent distribution to the blood (which many curcuminoid supplements and delivery mechanisms attempt to encourage or enhance), the present curcuminoid complexes are protected and surrounded by the phosphatidylcholine of the complex, as well as a triglyceride matrix of the supplement, facilitating absorption of the curcuminoid into chylomicrons for uptake into the lymphatic system via lacteals for circulation through the body, bypassing first-pass liver metabolism of curcumin and omega-3 phospholipids. By being incorporated into chylomicron particles, curcuminoids are available to be transferred to cells by diffusion into cellular phospholipid membranes, wherein the curcuminoids locate inside the phospholipid bilayer, similarly to the krill oil phospholipid interaction of the invention. This difference in absorption is made possible because of the complexing described herein. Such a different pathway to absorption avoids or minimizes the undesired metabolic processes that curcumin is typically subjected to (e.g., in the liver and elsewhere) that results in modification of curcumin into metabolized forms exhibiting low bioavailability, followed by excretion thereof, rather than their absorption into the cellular membranes, where they may better be able to provide desirable benefits to the user.

In an embodiment, the capsule which contains the supplement is a hard or rigid capsule, formed from cellulose and/or gelatin, without any free glycerin, sorbitol or similar polyol plasticizers that would interfere with formation of the desired complex. For example, the capsule may be a rigid LiquidCap, rather than a softgel capsule.

The complex may include phosphatidylcholine and curcuminoids in a molecular or molar ratio of approximately 1:1, e.g., where the curcuminoid molecule is “wrapped” within the long omega-3 alkylene chains of the phospatidylcholine. This structure may be held together by weakly attractive Van der Waals and hydrogen bonding forces, in the absence of other free polyols (e.g., glycerin) that would interfere with such attractions. The weight ratio of curcuminoids to krill oil phosphatidylcholine may be from 0.1:1 to 5:1, greater than 1:1, such as greater than 1.25:1, by weight.

The krill oil may include a blend of phosphatidylcholines having carbon chain lengths of greater than 18 carbon atoms (e.g., 20 and 22 carbon atom chain lengths as in EPA and DHA). The krill oil may also include a substantial fraction of triglycerides, which serves as a matrix in which the PC-curcuminoid complexes can be dispersed. Importantly, the complexes are not present as liposomes, formed from scores or hundreds of oriented phospholipids, defining an encapsulated (aqueous) compartment at the center of the liposome spherule. Rather, in the present PC-curcuminoid complexes, the desired structure may include far fewer molecules, e.g., one curcuminoid molecule “wrapped” within a PC molecule. In other words, the omega-3 fatty acid moieties of the PC component of the krill oil may “wrap” around the curcuminoid, where such structure is maintained by weak Van der Waals and/or hydrogen bonding, and such complexes are dispersed within the triglyceride oily matrix component also provided by the krill oil.

The supplement may be substantially void of water, as aqueous environments interfere with the formation of the desired phosphatidylcholine-curcuminoid complex, and also contribute to the instability and degradation of curcuminoids in their desired free form (as opposed to the metabolized forms often found in blood and body tissues, which exhibit little to no bioactivity).

The supplement may further include medium-chain triglyceride (“MCT”) oil (e.g., triglycerides having a carbon chain length of 6-12 carbon atoms). Such MCT oil may improve bioavailability and/or stability of the components provided in the supplement. MCT oil is typically present in approximately a 1:1:1 weight ratio with krill oil and curcumin. The weight ratio of MCT oil to krill oil may be from 0.1:1 to 5:1, preferably less than 1:1, such as greater than 0.6:1, by weight.

If it is desired to modify the viscosity of the oil supplement, a viscosity modifier such as silica (e.g., fumed silica) may be added in a very small amount (e.g., less than 1%, less than 0.5%, less than 0.1%, or the like).

In addition, the phosphatidylcholine-curcuminoid complex in a triglyceride matrix can be protected by an astaxanthin antioxidant normally occurring in krill oil, as opposed to a liposomal or particulate type complex, as it is desired that the curcuminoid be freely soluble in the krill oil without the formation of liposomes. The presence of the triglycerides in the krill oil, and saturated fat triglycerides in the MCT oil, in the absence of free glycerin or similar plasticizer aids in prevention of such liposomal formation.

Further features and advantages of the present invention will become apparent to those of ordinary skill in the art in view of the detailed description of preferred embodiments below.

### DETAILED DESCRIPTION

### I. Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising,” which is synonymous with “including,” “containing,” or “characterized by,” is inclusive or open-ended and does not exclude additional, unrecited elements or method steps.

The term “consisting essentially of” limits the scope of a claim to the specified materials or steps “and those that do not materially affect the basic and novel characteristic(s)” of the claimed invention.

The term “consisting of” as used herein, excludes any element, step, or ingredient not specified in the claim.

It must be noted that, as used in this specification and the appended claims, the singular forms “a”, “an” and “the” include plural referents unless the content clearly dictates otherwise. Thus, for example, reference to a “starch” includes one, two or more such starches.

Numbers, percentages, ratios, or other values stated herein may include that value, and also other values that are about or approximately the stated value, as would be appreciated by one of ordinary skill in the art. As such, all values herein are understood to be modified by the term “about”. A stated value should therefore be interpreted broadly enough to encompass values that are at least close enough to the stated value to perform a desired function or achieve a desired result, and/or values that round to the stated value. The stated values include at least the variation to be expected in a typical manufacturing process, and may include values that are within 10%, within 5%, within 1%, etc. of a stated value.

Some ranges may be disclosed herein. Additional ranges may be defined between any values disclosed herein as being exemplary of a particular parameter. All such ranges are contemplated and within the scope of the present disclosure.

In the application, effective amounts are generally those amounts listed as the ranges or levels of ingredients in the descriptions, which follow hereto. Unless otherwise stated, all percentages, ratios, parts, and amounts used and described herein are by weight.

The phrase “free of”, “void of” or similar phrases if used herein means that the composition or article comprises 0% of the stated component, that is, the component has not been intentionally added. However, it will be appreciated that such components may incidentally form thereafter, under some circumstances, or such component may be incidentally present, e.g., as an incidental contaminant.

The phrase “substantially free of”, “substantially void of” or similar phrases as used herein means that the composition or article preferably comprises 0% of the stated component, although it will be appreciated that very small concentrations may possibly be present, e.g., through incidental formation, contamination, or even by intentional addition. Such components may be present, if at all, in amounts of less than 1%, less than 0.5%, less than 0.25%, less than 0.1%, less than 0.05%, less than 0.01%, less than 0.005%, less than 0.001%, or less than 0.0001%. In some embodiments, the compositions or articles described herein may be free or substantially free from any specific components not mentioned within this specification.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

### II. Introduction

The present invention relates to supplements that include both krill oil, which includes a significant fraction of phosphatidylcholine (PC), in combination with one or more curcuminoids, where the krill oil and one or more curcuminoids are present as a weakly non-covalently bonded complex (e.g., held together with Van der Waals forces and/or hydrogen bonding), where the supplement is packaged in a hard capsule that is not a softgel capsule, but in which the capsule is substantially void of free glycerin and other polyol plasticizers. Rather, the supplement may be packaged in a LiquidCap, which is a rigid capsule formed from gelatin and/or cellulose, without any plasticizer (e.g., free glycerin).

A related embodiment is directed to a method for reducing inflammation, comprising administering (e.g., orally) a supplement as described above, to an individual, in order to treat inflammation (e.g., joint inflammation, such as inflammation of joints associated with one or more of knees, hips, the spine, or other joint). Such supplements may also be used to treat delayed onset muscle soreness from exercise, exercise-induced muscle damage from over-exercise, muscular pain after intense exercise, musculotendinous pain after strenuous exercise (endurance and/or resistance exercise), and/or joint pain after intense strenuous exercises. In addition to such various treatments, the present supplements may be useful for improvements in mood, cognition, cardiovascular functions (improved circulation, efficient cardiac performance/rhythm and other benefits of omega-3s and curcuminoids.

While existing formulations including krill oil and curcumin are available (e.g., from Swanson), such are packaged within a softgel, where the softgel includes glycerin in order to maintain the desired soft, flexible characteristics of the softgel capsule. Absent such glycerin, softgel capsules tend to crack, and exhibit even worse leakage. Even with the glycerin, existing formulations of krill oil and curcumin, packaged in softgel (e.g., as in Swanson's product) have a tendency to leak, which is undesirable in a consumer product. As a result, there is significant waste and lost production runs, due to leaking capsules being unusable. In addition, while curcumin promises many health benefits, the existing formulations have not effectively addressed problems that lead to low absorption, uptake and bioavailability.

By eliminating use of softgels, and eliminating use of free glycerin or similar polyol plasticizers in general, and packaging krill oil in a liquid capsule that is not a softgel and does not include glycerin, many of these problems are surprisingly addressed, leading to a product that is more stable, less prone to leakage, and exhibits improved absorption, uptake and bioavailability. In the absence of free glycerin, the phosphatidylcholine of the krill oil and the curcumin (or other curcuminoids) form a weakly bonded complex held together by Van der Waals forces and hydrogen bonding. Such weak attractions are actually destroyed in the presence of glycerin or a similar polyol plasticizer, due to the presence of the —OH moieties of the free glycerin, which interfere with the formation of such a complex.

### III. Krill Oil and Curcuminoid Components

As noted, krill oil includes phosphatidylcholines. An exemplary 2-dimensional structure for phosphatidylcholine is shown below.

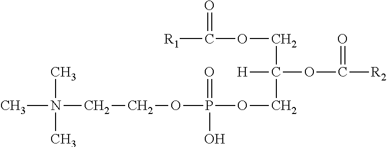

The R₁ and R₂ groups shown in the above structure may be any of a wide variety of organic groups (e.g., particularly carbon chains). In the case of phosphatidylcholines present within krill oil, the R₁ and R₂ groups may be alkane or alkylene groups, having for example, 12 to 30, 14 to 26, 16 to 24, 18 to 24, or 20 to 22 carbon atoms. A specific example of a phosphatidylcholine (specifically 1-palmitoyl-2-linoleoylphosphatidylcholine) is shown below.

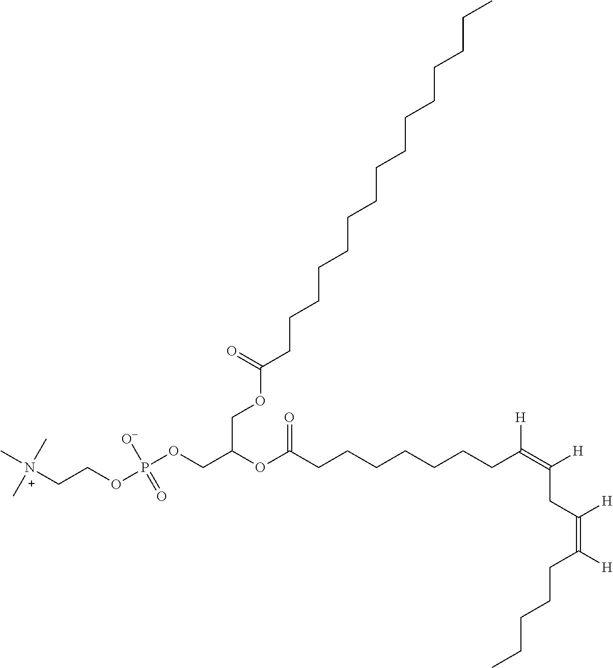 [SMILES: CCCCC/C=C\C/C=C\CCCCCCCC(=O)OC(COC(=O)CCCCCCCCCCCCCCC)COP(=O)([O-])OCC[N+](C)(C)C]

In particular, in some embodiments, the R1 and/or R2 groups may be configured to provide the phosphatidylcholine with omega-3 fatty acid groups, where the R1 and/or R2 group is a polyunsaturated organic carbon chain including a C═C double bond, three atoms away from the terminal methyl group. Such a fatty acid group may have significant polyunsaturation, e.g., far more than just a single unsaturated C═C bond. For example, in an embodiment, the fatty acid group may include at least 3, at least 4, or at least 5 unsaturated C═C bonds. EPA and DHA include 5 and 6 such unsaturated C═C bonds, respectively. EPA and DHA are specific examples of such omega 3-fatty acids. DHA has the structure shown below.

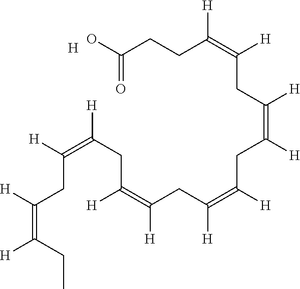

EPA has the structure shown below.

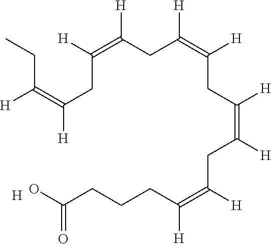

In an embodiment, R₁ and/or R₂ are alkylene chains that provide the phosphatidylcholine with an EPA and/or DHA fatty acid moiety. In an embodiment, the R₁ and R₂ structures may be the same, or different. In an embodiment, one of R₁ and R₂ can be EPA, while the other of R₁ and R₂ is DHA. It will be apparent from the structures described for the phosphatidylcholine, that the fatty acid chains are flexible, with the ability to “wrap” around a curcuminoid molecule, forming a protective complex therewith as described herein.

As used herein, “curcuminoids” and “free curcuminoids” refer to curcumin, demethoxycurcumin (where 1 methoxy group has been removed from curcumin), and bisdemethoxycurcumin (where both methoxy groups have been removed from curcumin). Curcuminoids are linear (rather than cyclic) diarylheptanoids, including two aromatic rings (aryl groups) joined together by a seven carbon chain. Other derivatives of curcumin may also be encompassed by “curcuminoids”, although “curcuminoids” as used herein excludes metabolic or other derivatives of curcumin (e.g., glucuronidated, sulfated, methylated, and/or acetylated) that exhibit poor bioavailability.

Curcumin has the structure shown below.

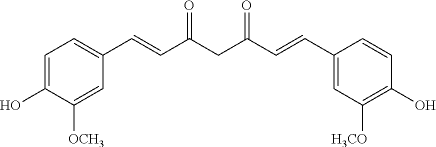 [SMILES: COc1cc(/C=C/C(=O)CC(=O)/C=C/c2ccc(O)c(OC)c2)ccc1O]

Demethoxycurcumin has the structure shown below.

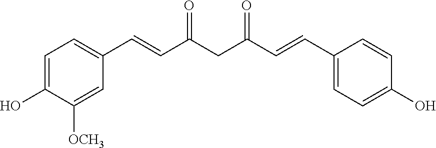 [SMILES: COc1cc(/C=C/C(=O)CC(=O)/C=C/c2ccc(O)cc2)ccc1O]

Bisdemethoxycurcumin has the structure shown below.

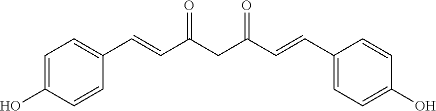 [SMILES: O=C(/C=C/c1ccc(O)cc1)CC(=O)/C=C/c1ccc(O)cc1]

Curcumin and the other described curcuminoids have a relatively rigid structure, in the form of a twisted ribbon L-shape, as shown below.

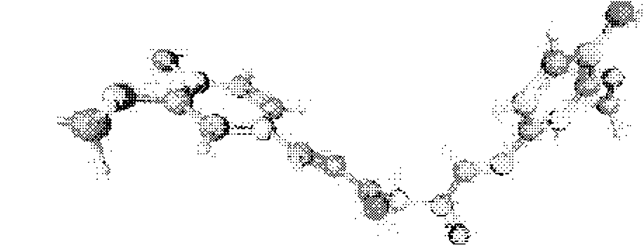

As described herein, the flexibility of the phosphatidylcholine molecule allows the sn-2 fatty acid (usually EPA or DHA) portion of such to wrap around the curcuminoid molecule, attracted by Van der Waals forces between the polyunsaturated benzene rings of the curcuminoid and the polyunsaturated fatty acid chains of the phosphatidylcholine. Each complex may be surrounded by a matrix of triglycerides that help maintain the curcuminoid:phosphatidylcholine complex. Particularly where the chains of the phosphatidylcholine are relatively long (e.g., greater than 18 carbons long) the omega-3 fatty acid portions of the phosphatidylcholine molecule can have a nearly circular shape so as to wrap around the curcuminoid, facilitating close proximity of the polyunsaturated bonds of the PC molecule to the double bonds in the curcuminoid.

### IV. Some Key Relevant Facts

Fact: curcuminoids are fat-soluble and are easily degraded when exposed to aqueous solutions.

Fact: curcuminoids are soluble in krill oil, which is approximately half fat (triglycerides) and half phospholipids (e.g., particularly phosphatidylcholine).

Fact: phospholipids are soluble in both aqueous and oily conditions.

Fact: krill oil phospholipids are rich in the omega-3 fatty acids EPA and DHA, and are similar or identical in composition or structure to phospholipids in human cell membranes.

Fact: combining krill oil with curcuminoids in softgels presents difficulties in manufacture (e.g., leaking softgels, wasted production runs, missed order fulfillments, excess cost, etc.).

Fact: glycerol (glycerin) is commonly used for production of softgels, but not for LiquidCaps. The terms glycerin and glycerol are used interchangeably herein.

Fact: LiquidCaps (hard gelatin or cellulosic two-piece, banded capsules) are glycerol-free and do not have the production problems that softgels encounter.

Fact: free curcuminoids have desirable bioactivity, as evidenced by numerous animal and in vitro studies, although to now, it has been difficult to achieve a supplement that could deliver curcuminoids in such free form, leading to problems with absorption, uptake and bioavailability.

Fact: modified and degraded forms of curcuminoids such as available through current supplements do not have desired or efficacious bioactivity.

Fact: curcuminoids are poorly absorbed, have low bioavailability and are extensively modified and degraded into ineffective or less efficacious forms by current means of delivery, with no or only tiny, inefficacious amounts of free curcuminoids being delivered to desired target tissues.

Fact: absorption claims for curcuminoid materials are misleading and inaccurate about delivering free curcuminoids to tissues due to technical practices.

Fact: preliminary human evidence has found somewhat improved efficacy for krill oil:curcuminoid softgels that was not found with other “enhanced-absorption” curcumin materials.

Fact: krill oil:curcuminoid softgels are not an ideal delivery system because of manufacturing issues but more importantly, because glycerol interferes with the formation of a krill oil:curcuminoid complex, decreasing protection of curcuminoids and reducing potential efficacy.

Fact: unexpectedly, LiquidCaps allow maintenance of a krill oil:curcuminoid complex, that is easier to manufacture and that has better efficacy.

As described herein, a combination of krill oil with curcuminoids free of glycerol and filled into LiquidCaps has unique and unexpected advantages over other forms of krill oil:curcuminoids delivery.

### V. Characteristics of Exemplary Supplements

The existence of a 1:1 curcuminoid:PC complex has not been described in the scientific literature or the patent literature, to Applicant's knowledge, and is novel and provides unexpected benefits. Curcuminoids have not delivered their promised health benefits when orally ingested in humans and other animals that in vitro and animal (rodent) studies have reported. The claimed complex delivers free curcuminoids intact to cell membranes, enhancing the beneficial health effects of curcuminoids as well as omega-3 fatty acids.

Curcuminoids blended with krill oil rich in phospholipids administered packaged within a LiquidCap (hard gelatin or vegetable fiber capsules) uniquely deliver both curcuminoids and omega-3 fatty acids directly to cell membranes without metabolic transformations, increasing the cell membrane content of curcuminoids, which thus enhances desired biological effects (anti-inflammatory response, antioxidant benefits, and membrane stabilization) that deliver health benefits.

Krill oil rich in phospholipids (e.g., Superba2™ from Aker BioMarine), is unexpectedly attracted to curcuminoids, forming a unique 1:1 molecular combination complex between an omega-3-containing phospholipid and a curcuminoid that shields and protects the curcuminoid from degradative processes that otherwise occur inside dietary supplement products (oxidation or other degradation triggered by air, light, and/or water) and/or inside the human body (aqueous solutions at pHs that degrade curcuminoids spontaneously, as well as Phase I metabolic processes (e.g., reduction, oxidation, and/or hydrolysis, to name a few) and Phase II metabolic processes (e.g., addition of hydrophilic groups to a molecule resulting from Phase I or otherwise, such as conjugation reactions, glucuronidation, acetylation, methylation, and/or sulfation, to name a few).

Krill phospholipids and curcuminoids have a unique attraction when physically combined and mixed together in the absence of glycerin, preferably with an amount of triglycerides substantially equivalent to the amount of phospholipids, as provided within the desired sources of krill oil. The rigid 3-dimensional structure of curcumin combines with the flexible shape of omega-3 phospholipids to form a mutually protective complex that has the attractive strength to target fat-soluble nutrient absorption and distribution pathways, preventing or minimizing degradation from normal bodily conditions, and also having an easy, diffusible release inside cell membranes, which are phospholipid bilayers and the major active site of both curcuminoids and long-chain omega-3 fatty acids for anti-inflammatory properties.

The oily (triglyceride) component of krill oil helps keep individual curcumin molecules attracted to individual phosphatidylcholine (PC) phospholipid molecules. The molecular-scale flexible shape of PCs and the rigid molecular shape of curcuminoids (a twisted ribbon L shape as illustrated above) are kept in alignment to have both hydrophobic areas (exhibiting Van der Waals forces) and hydrophilic areas (exhibiting hydrogen-bonding between oxygens and hydroxyl groups on curcuminoids and PCs) that attract each other to form a non-covalently bonded, 3-dimensional “cage” to entrap a curcuminoid molecule within a PC molecule.

The flexibility of the PC molecule allows the sn-2 fatty acid (usually EPA or DHA) to wrap around a curcumin molecule, attracted by Van der Waals forces between the polyunsaturated benzene rings of curcuminoids and the polyunsaturated fatty acid chains. Each complex may have a sphere of triglycerides or otherwise be surrounded by triglycerides that helps maintain the curcuminoid:PC complex. Long-chain (greater than 18 carbons long) omega-3 fatty acids have a nearly circular shape, facilitating close proximity to the double bonds in curcuminoids. The sn-1 fatty acid (usually a saturated palmitate (C₁₆)) of omega-3 phospholipids has a generally linear shape that would maintain the complex's position around triglycerides in krill oil. Once inside a cell membrane, the sn-1 saturated fatty acid rotates to stay next to other fatty acids inside the cell membrane. This change in molecular location of the saturated fatty acid in krill oil phospholipids enables release of the curcuminoid from the curcuminoid:PC 1:1 complex, keeping curcuminoids inside the cell membrane. Typically, omega-3 phospholipids in cell membranes (e.g., in humans), have a palmitic group in the sn-1 position and an omega-3 or omega-6 in the sn-2 position. This is also the major configuration present in krill oil phospholipids. The sn-2 fatty acids are used by specific enzymes to form eicosanoids, etc. Lysophospholipids (minus a fatty acid) are formed, which are removed since they are very good detergents and disrupt membrane integrity. The sn-1 saturated fatty acids moiety sticks out in a long, linear chain, where they line up with other sn-1 saturated fatty acids to help form cell membranes (through Van der Waal forces). Such interactions also help keep krill oil a liquid oil at typical temperatures.

The placement of keto-form oxygens (i.e., oxygens of a ketone group R₁—(C═O)—R₂) and hydroxyl groups on the ends and the hinge point of the L-shape of curcuminoids allows interactions via hydrogen-bonding with the oxygens on the bound glycerol, hydroxyl and phosphate groups of phospholipids (as opposed to any free glycerol within the formulation). Furthermore, the hydrophobic area of curcuminoid molecules—the benzene rings and their polyunsaturated bridge that connects the two rings—is attracted by Van der Waals forces to omega-3 fatty acids. The two non-covalent molecular attraction modes keep curcuminoid molecules oriented in a favorable position to form the 1:1 curcuminoid:PC complex described herein. As explained, such a complex cannot form in the environment provided within existing softgel capsules, because the presence of free glycerin in such environment interferes with complex formation.

LiquidCaps are an integral and unique part of the ability to achieve and maintain the described curcuminoid-krill oil complex. The hydrogen-bonding aspect of the curcuminoid:PC complex also occurs with free glycerol, which interferes with and disrupts the hydrogen-bonding between krill oil phospholipids and curcuminoids, and thus, it is advantageous to exclude free glycerol (glycerin) from the mixture, and from the capsule. Softgels require free glycerol for their manufacture and long-term stability, and thus are not compatible with maintenance of the desired curcuminoid:PC complex. LiquidCaps do not contain glycerol and thus facilitate formation and maintenance of the curcuminoid:PC complex.

Astaxanthin normally present in krill oil (which accounts for the red color) is a potent antioxidant that does not degrade into harmful molecular species, unlike other fat-soluble antioxidants (e.g., beta carotene and/or tocopherols), protecting both the omega-3 fatty acids and curcuminoids from oxidative degradation. In an embodiment, the formulation may be substantially free of such undesireable antioxidants. Like astaxanthin, curcuminoids are potent antioxidants, also exhibiting the ability to keep the highly polyunsaturated omega-3 fatty acids (primarily EPA+DHA) from oxidative degradation. Each antioxidant protects the omega-3 fatty acids and curcuminoids from oxidative damage to preserve the 1:1 curcuminoid:PC complex. The mutually protective antioxidants keep more curcuminoids and omega-3 fatty acids available for delivery to cell membranes from oral intake of the formulation packaged within a LiquidCap.

In an embodiment, the supplement may include phosphatidylcholine and curcuminoids in a molecular or molar ratio of approximately 1:1. More generally, the weight ratio of curcuminoids to phosphatidylcholine may be from 0.1:1 to 5:1, greater than 1:1, such as greater than 1.25:1, by weight.

Furthermore, this combination provides an enhanced, targeted delivery system for delivery to cell membranes of active, free curcuminoids and omega-3 fatty acid phospholipids (omega-3 PCs). The resulting increase in cell membrane free curcuminoids and omega-3 phospholipids deliver antioxidant and anti-inflammatory actions to cells in need of those actions. The result is greater anti-inflammatory and antioxidant actions that manifest in improved cellular tissue health and reduced tissue damage, allowing improved tissue healing from normal wear and tear, exercise stress and autoimmune disorders (which are all pro-inflammatory). Other benefits may potentially include, but are not limited to cancer prevention, cancer treatment, improved brain health (including lower risk of Alzheimer's disease) and mitigation of other conditions in which inflammation plays a role.

The ability to deliver both curcuminoids in free forms and omega-3 PCs in the described manner largely bypasses blood uptake by the portal blood system, and thus, bypasses first pass liver degradation of curcuminoids, which is the predominant fate of other “enhanced absorption” curcuminoid supplements, regardless of how much curcuminoids are delivered to the liver. Intestinal and liver Phase I and Phase II metabolism processes convert free curcuminoids into glucuronide, sulfate, acetylated or methylated versions, increasing their water solubility, and thus, increasing their removal from the body. Liver metabolism also converts curcuminoids to their reduced forms (tetrahydrocurcuminoids, hexahydrocurcuminoids and octahydrocurcuminoids), which are further modified by glucuronidation, sulfation, acetylation or methylation, resulting in virtually zero free curcuminoids or free, reduced curcuminoids after oral administration of curcuminoids from dry powders and from existing enhanced-absorption preparations. Such modified curcuminoid metabolites are either devoid of biological activity or possess extremely low potency compared to the free curcuminoids contemplated herein. Modified curcuminoids are excreted with bile and/or urine, and are thus very limited for affecting health or having benefits for consumers.

One drawback of extant research on curcuminoid bioavailability in humans is the use of plasma and/or serum as the tissue or bodily fluid measured. Early research found that cellular uptake of any form of free curcuminoids was low or absent in such environments, including erythrocytes and white blood cells circulating in the bloodstream. Nevertheless, researchers have continued to focus on plasma and/or serum as the pathway and environment of choice for measuring curcuminoid uptake. This allows comparisons to be made among curcuminoid materials. More recently, some measurements of curcuminoid material uptake into erythrocytes have occurred, as well as measurement of fluorescent curcuminoids in retinal blood flow. Because of measurement methodology, it is not clear if the curcuminoids were free or modified. Animals, especially rodents, have quite different uptake mechanisms for curcuminoids compared to humans, showing large percentages of orally-administered free curcuminoids as free curcuminoids in plasma or serum.

Thus, animal results of absorption or efficacy for oral administration of curcuminoids are not particularly applicable to humans, who do not show the same significant concentrations of free curcuminoids after oral ingestion. Likewise, adding curcuminoids to in vitro cell or tissue cultures utilizing solvents such as DMSO or detergents to solubilize curcuminoids, combined with administration of concentrations that are hundreds or thousands of times greater than even “total curcuminoids” levels in human plasma or serum renders such in vitro results largely irrelevant for evaluating efficacy. Thus, it would be helpful to measure curcuminoids in actual human tissue samples in order to ascertain absorption, uptake and ultimately, efficacy of curcuminoids for humans.

The methodological practice of measuring and reporting numerous possible curcuminoid species in human blood or tissue samples has almost universally involved reporting such measurements after collecting the curcuminoid metabolites and reversing the modifications of glucuronidation, sulfation, and methylation to release free curcuminoids (including reduced curcuminoids). This is typically accomplished by treating biological samples with commercially available enzymes that remove the modifications. The resulting free curcuminoids are extracted from biological samples with solvents and the free curcuminoids are typically measured by various chromatographic methods. The reported results are reported as “curcumin,” or “total curcumins” or a similar appellation that is not a true reporting of levels of free curcuminoids actually present in such biological samples. Using the term “curcumin” or “curcuminoids” or even “total curcumin” to describe bioavailability and uptake study results is misleading, leading a reader to believe that significant free form curcumin was present in the biological fluids, with promise of biological activity. This is incorrect, as free curcumin can be measured by solvent extraction and measurement by chromatography without treatments to convert metabolized curcuminoids back into free curcuminoids. When true free curcumin is correctly measured and reported alongside “total curcuminoids,” free curcumin/curcuminoids are invariably extremely low or absent in human blood or tissues, far below levels found in animal and in vitro settings, which exhibit far higher free curcuminoid levels.

As stated previously, the primary circulating forms of curcuminoids in plasma and/or serum after oral ingestion are metabolized derivatives that have been shown to have little to no biological activity. While it is possible that localized inflamed tissue sites that have extracellular beta-glucuronidase activity may reverse some curcuminoid glucuronides (having little to no bioactivity) to free curcumin (which would exhibit significant bioactivity), such is unlikely, as this would expose free curcuminoids to an aqueous environment known to rapidly degrade curcuminoids into other compounds (including vanillin and/or ferulates) that do not have the same biological activity as free curcuminoids. Likewise, the reduced free curcuminoids, if their metabolic modifications were reversed locally, have lower biological activity than “virgin” curcuminoids and have not been shown to have similar or greater efficacy than free curcuminoids that have not undergone such modifications.

Most “enhanced absorption” methods or formulations intended to increase curcuminoid absorption include detergents or solubilizing agents that are intended to increase the water solubility of curcuminoids to increase concentration of such species in plasma and/or serum. This practice simply provides the intestinal epithelium and the liver with more curcuminoids for faster metabolism (much of which has already been glucuronidated or sulfated by intestinal epithelial cells). Healthy liver function has considerable ability to modify polyphenols, including curcuminoids, with the result that little, if any, free curcumin is present in circulation. Thus, in an embodiment, the present formulations are free of any such detergents or solubilizing agents.

Some “enhanced absorption” products include phospholipids present in a dry, solid form (examples are Longvida® and Meriva®). However, the dry form and type of phospholipids (usually soy, rich in relatively shorter linoleate (C₁₈) fatty acids or as saturated fatty acids) are not equivalent to omega-3 PCs in krill oil for their molecular shapes, and do not have the opportunity to form a krill oil-curcumin complex as described herein because of their methods of preparation, lack of oil and presence of other interfering substances (protein, fiber and free fatty acids) that would reduce or prevent formation of krill oil-curcumin complexes. Higher concentration of “total curcuminoids” has been reported from such materials in plasma and/or serum as compared to curcuminoid powder, but, as noted above, actual free curcumin levels (if reported at all) were well below therapeutic levels. Thus, the phospholipids in krill oil in the present invention are significantly different from other curcuminoid preparations containing phospholipids, which do not have the ability to form the krill oil-curcumin complexes described herein. In an embodiment, the phospholipids are not present in a dry or powdered form, but as a liquid oil. In addition, in an embodiment, the present formulations include omega-3 components, rather than other unsaturated components (such as linoleates, which are omega-6 components) to facilitate efficacious benefits.

Applicant's contemplated krill oil-curcumin complex bypasses the first-pass intestinal and liver metabolic modifications by going through the lymphatic system of fat delivery to the bloodstream. By depositing both curcuminoids and omega-3 PCs together into cell membranes, the exposure and diffusion of curcuminoids (which are hydrophobic) into plasma and/or serum is advantageously inhibited, and allows cell-cell transfer via cell membranes, in company with omega-3 phospholipids. Thus, measuring serum and/or plasma levels of curcuminoids in humans or animals after ingestion of the krill oil-curcumin complex described herein would be pointless and misleading, as the present uptake pathway does not rely on blood serum or plasma as part of the uptake pathway. Measurement of erythrocyte and other relevant biological tissue levels without enzymatic treatment of any metabolized curcuminoids exhibiting low bioavailability (e.g., glucuronidated, sulfated, methylated, and/or acetylated metabolized forms, or tetrahydrocurcuminoid, hexahydrocurcuminoid or octahydrocurcuminoid metabolized forms) might be feasible to show uptake of free curcumin after ingestion of the krill oil-curcumin complex, but the timing and preparation of sample collections would be critical. Endogenous and exogenous phospholipid omega-3s are transferred as described herein, and because the present disclosure provides curcuminoids complexed with phospholipid omega-3s, both the curcuminoids and the phospholipid omega-3s as described herein (in the form of a complex) are transported and delivered in a similar manner to cell membranes (e.g., of erythrocytes or other target cells). Such delivery provides for enhanced bioactivity as compared to existing alternatives.

### VI. Examples and Experimental Results

### Example 1

An exemplary formulation includes 500 mg of krill oil, and 300 mg of curcuminoids, packaged within a LiquidCap capsule, free of glycerin, free of detergents or solubilizing aids, etc. Such a formulation may include 250 mg of phosphatidylcholine phospholipids, 28 mg of DHA, 60 mg of EPA, 300 mg of triglycerides, and 0.05 mg of astaxanthin. The LiquidCap may be topped off with MCT (e.g., 200 mg of MCT). Viscosity of the formulation in the LiquidCap may be increased with the addition of a small amount (e.g., 20 mg) of fumed silica. Any of the above values may be varied, e.g., by ±50%, ±30%, ±20%, ±10%, or ±5%. The LiquidCap is formed from rigid gelatin or rigid cellulose, without the use of glycerin or similar plasticizers, to create a relatively rigid hard capsule, rather than a softgel (which capsules are soft and flexible). The formulation and capsule packaging provides an environment where the phsphatidylcholine and curcuminoids are able to form a complex, held together by weak Van der Waals and hydrogen bonding attractions, without the formation of strong covalent or ionic chemical bonds between the components of the complex, as described herein.

Another exemplary formulation includes 300 mg of krill oil, and 100 mg of curcuminoids, packaged within a LiquidCap capsule, free of glycerin, free of detergents or solubilizing aids, etc. Such a formulation may include 250 or 120 mg of phosphatidylcholine phospholipids, 17 mg of DHA, 36 mg of EPA, 180 mg of triglycerides, and 0.03 mg of astaxanthin. The LiquidCap may be topped off with MCT (e.g., 400 mg of MCT). Viscosity of the formulation in the LiquidCap may be increased with the addition of a small amount (e.g., 20 mg) of fumed silica. Any of the above values may be varied, e.g., by ±50%, ±30%, ±20%, ±10%, or ±5%.

Without departing from the spirit and scope of the invention, one of ordinary skill can make various changes and modifications to the invention to adapt it to various usages and conditions. As such, these changes and modifications are properly and intended to be, within the full range of equivalence of the following claims.

1. A supplement comprising:
krill oil including phosphatidylcholine (PC); and
one or more curcuminoids;
wherein the krill oil and one or more curcuminoids are present as a weakly non-covalently bonded complex held together with Van der Waals forces and/or hydrogen bonding, wherein the supplement is packaged in a capsule that is not a softgel capsule, the capsule being substantially void of glycerin and other polyol plasticizers.

2. The supplement of claim 1, wherein the complex entraps the curcuminoid with a long-chain (longer than 18 carbons) omega-3 phosphatidylcholine phospholipid and shields the curcuminoid from adverse interactions with hydrophilic, aqueous and/or metabolic environments.

3. The supplement of claim 1, wherein the capsule is formed from cellulose and/or gelatin, without any glycerin, sorbitol or other polyol plasticizer.

4. The supplement of claim 1, wherein the capsule and contents of the capsule are substantially void of water.

5. The supplement of claim 1, wherein the capsule comprises a rigid LiquidCap capsule.

6. The supplement of claim 1, wherein the complex includes phosphatidylcholine and curcuminoids at a molar ratio of about 1:1.

7. The supplement of claim 1, wherein the krill oil includes a blend of phosphatidylcholine having carbon chain lengths of greater than 18 carbon atoms, and triglycerides.

8. The supplement of claim 1, wherein the supplement further comprises medium-chain triglyceride oil (MCT), with a carbon chain length of 6-12 carbons.

9. The supplement of claim 1, wherein the one or more curcuminoids comprise at least one of curcumin, demethoxycurcumin or bisdemethoxycurcumin.

10. The supplement of claim 1, wherein the one or more curcuminoids are substantially void of methylated, acetylated, sulfated, or glucuronidated curcumin, demethoxycurcumin or bisdemethoxycurcumin.

11. The supplement of claim 1, wherein the one or more curcuminoids are substantially void of tetrahydrocurcuminoid, hexahydrocurcuminoid and octahydrocurcuminoid.

12. The supplement of claim 1, wherein the supplement further comprises a viscosity modifier.

13. The supplement of claim 12, wherein the viscosity modifier comprises silica.

14. A method for reducing inflammation, comprising administering a supplement comprising:
krill oil including phosphatidylcholine (PC); and
one or more curcuminoids;
wherein the krill oil and one or more curcuminoids are present as a weakly non-covalently bonded complex held together with Van der Waals forces and/or hydrogen bonding, wherein the supplement is packaged in a capsule that is not a softgel capsule, the capsule being substantially void of glycerin and other polyol plasticizers.

15. The method of claim 14, wherein the method is used to treat joint inflammation.

16. The method of claim 15, wherein the method is used to treat inflammation of joints associated with one or more of knees, hips or spine.

17. The method of claim 14, wherein the complex includes phosphatidylcholine and curcuminoids at a molar ratio of about 1:1.

18. The method of claim 14, wherein a ratio of curcuminoids to krill oil phosphatidylcholine is from 0.1:1 to 5:1 by weight.

19. The method of claim 14, wherein a ratio of curcuminoids to krill oil phosphatidylcholine is greater than 1:1 by weight.

20. The method of claim 14, wherein a ratio of curcuminoids to krill oil phosphatidylcholine is greater than 1.25:1 by weight.

21-22. (canceled)
